(19) United States
(12) Patent Application Publication
(10) Pub. No.: US 2011/0083256 A1
(43) Pub. Date: Apr. 14, 2011

(54) Structure for Securing a Secondary Goggle Frame

(76) Inventor: Tzu-Feng Wang-Lee, Rende Township (TW)

(21) Appl. No.: 12/774,805

(22) Filed: May 6, 2010

(30) Foreign Application Priority Data
Oct. 9, 2009 (TW) TW 098218603

(51) Int. Cl. A61F 9/02

(52) U.S. Cl. 2/434

(57) ABSTRACT

A structure for securing a secondary goggle frame is provided. The structure is characterized by a connector configured for connecting the secondary goggle frame to a primary goggle frame. The primary goggle frame has a central nose support whose inner edge is formed with plural ribs for engaging with plural recesses on a side of the connector. The secondary goggle frame has an engaging section which can be inserted in a groove of the connector such that a fastening portion of the secondary goggle frame is fastened with a fastening hole of the connector. The components of the structure are simple and can be easily assembled to allow the secondary goggle frame fitted with vision correction lenses to be securely and stably positioned in the primary goggle frame.

### BACKGROUND OF THE INVENTION

1. Technical Field

The present invention relates to an improved structure for securing a secondary goggle frame. More particularly, the present invention relates to a structure configured for rapidly installing a secondary goggle frame to a pair of protective goggles, wherein the secondary goggle frame is fitted with vision correction lenses to provide the wearer of the protective goggles with a clear vision.

2. Description of Related Art

Commercially available protective goggles typically have a goggle frame bearing transparent or colored lenses and configured to fit the wearer's face when worn. However, those who have poor eyesight (e.g., those with myopia, hyperopia, presbyopia, astigmatism, etc.) must put on contact lenses before wearing the goggles. Since contact lenses are not suitable for everyone, an alternative is to put on an ordinary pair of glasses with vision correction lenses before wearing the goggles, which nevertheless subjects the wearer to the compression of two frames. Therefore, protective goggles with a secondary goggle frame fitted with vision correction lenses have been developed for those with poor vision so as to suit their practical needs and eliminate the inconveniences they may otherwise experience when using protective goggles.

### BRIEF SUMMARY OF THE INVENTION

The present invention provides a novel configuration for protective goggles with a secondary goggle frame. The secondary goggle frame is mounted with vision correction lenses and can be easily coupled to the protective goggles by means of a simple connector whenever needed.

The primary object of the present invention is to connect a secondary goggle frame to a primary goggle frame with a connector. The primary goggle frame has a central nose support whose inner edge is formed with a plurality of ribs for engaging with a plurality of recesses on a side of the connector. The secondary goggle frame has an engaging section which can be inserted in a groove of the connector such that a fastening portion of the secondary goggle frame is fastened with a fastening hole of the connector. These simple components can be easily assembled to allow the secondary goggle frame fitted with vision correction lenses to be securely and stably positioned in the primary goggle frame.

### BRIEF DESCRIPTION OF THE SEVERAL VIEWS OF THE DRAWINGS

The invention as well as a preferred mode of use, further objects, and advantages thereof will be best understood by referring to the following detailed description of an illustrative embodiment in conjunction with the accompanying drawings, in which:

FIG. 1 is an exploded perspective view of the present invention;

FIG. 1A is an enlarged view of the circled area A in FIG. 1;

FIG. 2 is an assembled perspective view of the present invention;

FIG. 3 is a sectional perspective view of the present invention; and

FIG. 3A is an enlarged view of the circled area A in FIG. 3.

### DETAILED DESCRIPTION OF THE INVENTION

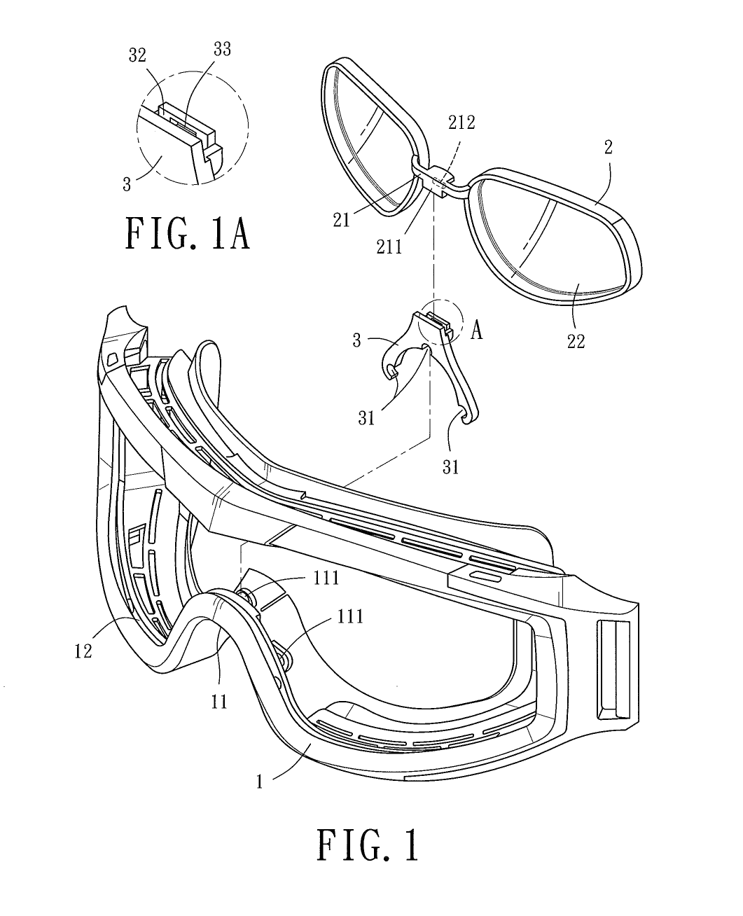

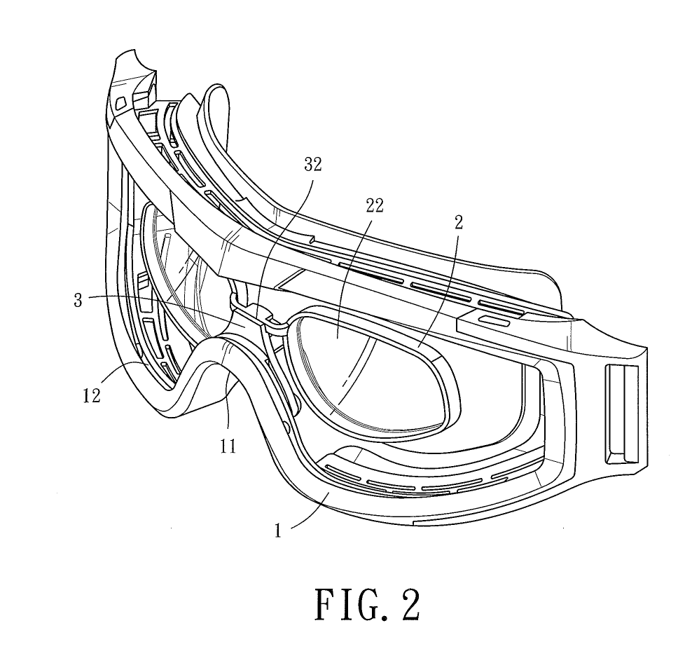

Referring to FIG. 1 and FIG. 2, according to the present invention, a structure for securing a secondary goggle frame includes a primary goggle frame 1 fitted with an ordinary lens, the secondary goggle frame 2 fitted with vision correction lenses 22, and a connector 3 for connecting the primary and secondary goggle frames 1 and 2.

The primary goggle frame 1 is a loop-shaped structure integrally formed with a nose support 11. The front inner rim of the primary goggle frame 1 is peripherally formed with a groove 12 in which an ordinary lens is fitted. The nose support 11, which is centrally provided on the primary goggle frame 1, has an inner edge formed with a plurality of ribs 111.

The secondary goggle frame 2 includes a central connecting section 21 bilaterally formed with frame units fitted respectively with the vision correction lenses 22, wherein the vision correction lenses 22 include those for correcting myopia, hyperopia, presbyopia, astigmatism, etc. An engaging section 211 extends downward from the connecting section 21 while a fastening portion 212 is protrudingly provided on a rear side of the connecting section 21.

The connector 3 has a side shaped according to the inner edge of the central nose support 11 of the primary goggle frame 1 and provided with recesses 31 corresponding in position to the ribs 111 of the primary goggle frame 1. In addition, the side of the connector 3 that corresponds in position to the secondary goggle frame 2 is formed with a groove 32. The groove 32 has a rear wall formed with a fastening hole 33.

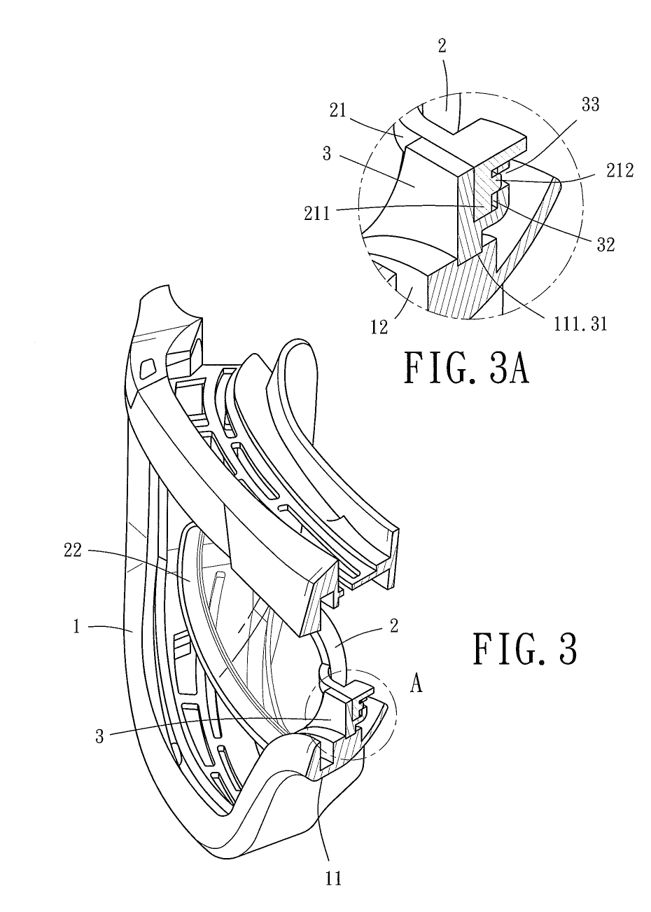

The primary goggle frame 1, the secondary goggle frame 2, and the connector 3 are assembled in the following manner. To begin with, referring to FIG. 1 and FIG. 3, the recesses 31 of the connector 3 are engaged with the ribs 111 of the primary goggle frame 1. Then, the engaging section 211 of the secondary goggle frame 2 fitted with the vision correction lenses 22 is inserted into the groove 32 of the connector 3 such that the fastening portion 212 of the secondary goggle frame 2 is fastened with the fastening hole 33 of the connector 3. Thus, by virtue of the connector 3, the secondary goggle frame 2 is conveniently, rapidly, and yet stably secured to the primary goggle frame 1.

What is claimed is:

1. A structure for securing a secondary goggle frame, the structure comprising a primary goggle frame fitted with an ordinary lens and the secondary goggle frame fitted with a vision correction lens, the structure characterized in that:
the primary goggle frame comprises a central nose support having an inner edge formed with a plurality of ribs for engaging with a plurality of recesses formed on a side of a connector, and the secondary goggle frame has an engaging section insertable in a groove of the connector such that a fastening portion of the secondary goggle frame is fastened with a fastening hole of the connector;
wherein the primary goggle frame, the secondary goggle frame, and the connecter can be easily assembled, thus allowing the secondary goggle frame fitted with the vision correction lens to be positioned stably and securely in the primary goggle frame.
